SEQUENCE LISTING (2 sequences)

SEQ ID NO: 1 - DNA, 34 bp, Artificial Sequence
  Synthetic oligonucleotide
ggatgccattggggatttcctctttactggatgt

SEQ ID NO: 2 - protein, 8 aa, Artificial Sequence
  Synthetic peptide
RKRTLRRL

(19) United States
(12) Patent Application Publication
(10) Pub. No.: US 2008/0279897 A1
(43) Pub. Date: Nov. 13, 2008

(54) Method of Treating Cellular Damage

(75) Inventors: Pu Xia, New South Wales (AU); Lijun Wang, New South Wales (AU); Mathew Alexander Vadas, New South Wales (AU)

Correspondence Address: SCULLY, SCOTT, MURPHY & PRESSER, P.C., 400 GARDEN CITY PLAZA, SUITE 300, GARDEN CITY, NY, 11530, US

(73) Assignee: MEDVET SCIENCE PTY. LTD., STEPNEY (AU)

(21) Appl. No.: 11/795,731

(22) PCT Filed: Jan. 17, 2006
(86) PCT No.: PCT/AU2006/000054

§ 371 Date: Nov. 30, 2007

(30) Foreign Application Priority Data
Jan. 21, 2005 (AU) 2005900257

(51) Int. Cl. A61K 39/10; C12N 5/00; A61K 31/131; A61P 3/10; A61K 38/45; A61K 31/661

(52) U.S. Cl. 424/240.1; 435/375; 514/669; 424/94.5; 514/114

(57) ABSTRACT

The present invention relates generally to a method of modulating hyperglycaemia-induced endothelial cell functioning and agents useful for same. More particularly, the present invention relates to a method of modulating hyperglycaemia-induced vascular endothelial cell functioning by modulating intracellular sphingosine kinase-mediated signalling. The method of the present invention is useful, inter alia, in the treatment and/or prophylaxis of the adverse vascular endothelial cell functioning associated with conditions characterised by hyperglycaemia, and/or diabetes mellitus, per se.

### FIELD OF THE INVENTION

The present invention relates generally to a method of modulating hyperglycaemia-induced endothelial cell functioning and agents useful for same. More particularly, the present invention relates to a method of modulating hyperglycaemia-induced vascular endothelial cell functioning by modulating intracellular sphingosine kinase-mediated signalling. The method of the present invention is useful, inter alia, in the treatment and/or prophylaxis of the adverse vascular endothelial cell functioning associated with conditions characterised by hyperglycaemia, and/or diabetes mellitus, per se.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Diabetes mellitus is characterised by an abnormality of carbohydrate metabolism resulting in elevated glucose levels in both the blood and the urine. The failure of the human body to properly metabolise the glucose is caused by defects in insulin secretion or use of insulin. Insulin is produced by β-cells in the islets of the pancreas and permits the body to utilise glucose as a source of energy. When this process cannot occur, the body compensates by utilising alternative sources of energy such as stored fats. However, this leads to rapidly rising levels of glucose and the accumulation of ketones in the bloodstream due to the occurrence of extensive fat metabolism.

Diabetes is broadly classified into two groups termed Type 1 diabetes and Type 2 diabetes. Type 1 diabetes (often referred to as juvenile onset diabetes due to its appearance in childhood or early adolescence) is a debilitating autoimmune condition caused by the selective destruction of insulin producing β-cells in the islets of the pancreas. Its onset is abrupt and occurs typically prior to the age of 20 years. Presently, however, Type 1 diabetes is increasingly presenting in adults. This disease is characterised by lack of β-cell function and no insulin production, and therefore insulin therapy is required. Type 2 diabetes, however, is characterised by insulin resistance, a condition in which the body fails to properly use insulin, which is often accompanied by obesity and other metabolic disorders. There are frequently no overt symptoms observed. Insulin secretory defects are evident very early in disease in both Type 1 and Type 2 diabetes, despite their differing aetiology.

Diabetic vascular complications, affecting both micro- and macro-blood vessels, represent major causes of disability and death in the patients with type 1 and type 2 diabetes. Diabetes is now recognized as a potent and independent risk factor for the development of coronary, cerebrovascular and peripheral atherosclerotic disease (Beckman et al., 2002, JAMA 287:2570-2581). Several large prospective clinical and epidemiological studies have also shown that intensive glycemic control can reduce the occurrence or progression of diabetic microvascular diseases (The Diabetes Control and Complications Trial Research Group. 1993 N. Engl. J. Med. 329:977-986; UK Prospective Diabetes Study (UKPDS) Group. 1998. Lancet 352:837-853), indicating a leading role for hyperglycaemia in causing vascular lesions. Thus, a better understanding of the mechanisms leading to vascular lesions by hyperglycaemia may have a significant impact on the therapeutic strategy for improvement of the clinical outcomes of diabetic patients. Accordingly, there is an ongoing need to elucidate the mechanisms by which the onset of a hyperglycaemic state effects cellular damage, in particular endothelial cell damage.

In work leading up to the present invention it has been determined that the adverse endothelial cell outcomes which are related to the onset of a hyperglycaemic state are, in fact, caused by hyperglycaemia mediated up-regulation in sphingosine kinase activity. The elucidation of this cellular signalling mechanism now facilitates the rational design of methodology directed to treating the adverse vascular functioning associated with conditions characterised by hyperglycaemia, such as diabetes.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. The subject specification contains nucleotide sequence information prepared using the programme PatentIn Version 3.1, presented herein after the bibliography. Each nucleotide sequence is identified in the sequence listing by the numeric indicator <210> followed by the sequence identifier (eg. <210>1, <210>2, etc). The length, type of sequence (DNA, etc) and source organism for each nucleotide sequence is indicated by information provided in the numeric indicator fields <211>, <212> and <213>, respectively. Nucleotide sequences referred to in the specification are identified by the indicator SEQ ID NO: followed by the sequence identifier (eg. SEQ ID NO: 1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing

One aspect of the present invention is directed to a method of modulating hyperglycaemia-induced endothelial cell functioning, said method comprising modulating the functioning of sphingosine kinase mediated signalling in said cell wherein down-regulating said sphingosine kinase signalling downregulates said endothelial cell activity

Another aspect of the present invention more preferably provides a method of modulating hyperglycaemia-induced vascular endothelial cell functioning, said method comprising modulating the functioning of sphingosine kinase mediated signalling in said cell wherein down-regulating said sphingosine kinase signalling down-regulates said vascular endothelial cell activity.

Yet another aspect of the present invention provides a method of down-regulating hyperglycaemia-induced vascular endothelial cell functioning, said method comprising modulating the functioning of sphingosine kinase mediated signalling in said cell.

Still another aspect of the present invention is directed to a method of down-regulating hyperglycaemia-induced vascular endothelial cell dysfunction, said method comprising down-regulating the functioning of sphingosine kinase mediated signalling in said cell.

In a related aspect the present invention is directed to a method of modulating hyperglycaemia-induced endothelial cell functioning in a mammal, said method comprising modulating the functioning of sphingosine kinase mediated signalling in said mammal wherein down-regulating sphingosine kinase signalling down-regulates said endothelial cell activity.

A further aspect of the present invention provides a method of modulating hyperglycaemia-induced vascular endothelial cell functioning in a mammal, said method comprising modulating the functioning of sphingosine kinase mediated signalling in said mammal wherein down-regulating sphingosine kinase signalling down-regulates said vascular endothelial cell activity.

Another further aspect of the present invention provides a method of down-regulating hyperglycaemia-induced vascular endothelial cell functioning in a mammal, said method comprising down-regulating the functioning of sphingosine kinase mediated signalling in said mammal.

Yet another further aspect of the present invention is directed to a method for the treatment and/or prophylaxis of a condition in a mammal, which condition is characterised by aberrant, unwanted or otherwise inappropriate hyperglycaemia-induced endothelial cell functioning, said method comprising modulating the functional activity of sphingosine kinase mediated signalling in said cell wherein down-regulating sphingosine kinase signalling down-regulates said endothelial cell activity.

Still another further aspect of the present invention is directed to a method for the treatment and/or prophylaxis of a condition in a mammal, which condition is characterised by aberrant, unwanted or otherwise inappropriate hyperglycaemia-induced vascular endothelial cell functioning, said method comprising modulating the functional activity of sphingosine kinase mediated signalling in said cell wherein down-regulating sphingosine kinase signalling down-regulates said vascular endothelial cell activity.

Yet still another further aspect of the present invention is directed to a method for the treatment and/or prophylaxis of a condition in a mammal, which condition is characterised by aberrant, unwanted or otherwise inappropriate hyperglycaemia-induced vascular endothelial cell functioning, said method comprising down-regulating the functional activity of sphingosine kinase mediated signalling in said cell.

The present invention further provides a method for the treatment and/or prophylaxis of a symptom of diabetes, which symptom is characterised by aberrant, unwanted or otherwise inappropriate hyperglycaemia-induced vascular endothelial cell functioning, said method comprising down-regulating the functional activity of said sphingosine kinase mediated signalling in said cell.

Another aspect of the present invention relates to the use of an agent capable of modulating sphingosine kinase mediated signalling in the manufacture of a medicament for the regulation of hyperglycaemia-induced endothelial cell functioning in a mammal wherein down-regulating sphingosine kinase signalling down-regulates said endothelial cell activity.

Still another aspect of the present invention relates to the use of an agent capable of modulating sphingosine kinase mediated signalling in the manufacture of a medicament for the regulation of hyperglycaemia-induced vascular endothelial cell functioning in a mammal wherein down-regulating sphingosine kinase signalling down-regulates said vascular endothelial cell activity.

Yet another aspect of the present invention relates to the use of an agent capable of down-regulating sphingosine kinase mediated signalling in the manufacture of a medicament for the regulation of hyperglycaemia-induced vascular endothelial cell functioning in a mammal.

In yet another further aspect, the present invention contemplates a pharmaceutical composition comprising the modulatory agent as hereinbefore defined together with one or more pharmaceutically acceptable carriers and/or diluents.

### BRIEF DESCRIPTION OF THE DRAWINGS

FIG. 1 is a graphical representation of the effect of hyperglycaemia on SphK activity in vivo. SphK activity was measured in aorta and heart from age-matched controls (Cont), STZ-induced diabetic rats (DM) or in diabetic rats treated with insulin (DM+Ins). Data are mean ±SEM (n=7; number of rats). *p<0.05; †p <0.01.

FIG. 2 is a graphical representation of the effect of high glucose on SphK activity in endothelial cells. SphK activity (A) and S1P formation (B) were measured in HUVEC (grey bars) and BAEC (dark bars) exposed to 5.5 mM glucose (NG), 22 mM glucose (HG), NG+16.5 mM mannitol (Mtol) or NG+16.5 mM L-glucose (L-glu) for 3 days as described in ‘Methods’. Data are mean ±SEM (n=3 to 5). *p<0.01 versus NG.

FIG. 3 is a graphical representation of the effect of SphK on high glucose-induced adhesion molecule expression by endothelial cells. Confluent monolayers of HUVEC were incubated for 3 days with 5.5 mM (NG), 22 mM (HG) glucose or HG plus 2.5 μM DMS (HG+DMS). Then, (A) the cell surface expression of adhesion molecules was assayed by flow cytometry, and (B) SphK or PKC activity was measured as described in ‘Methods’. Data are mean ±SEM (n=4 to 6). *p<0.01 versus 5.5 mM glucose (panel A). †p <0.01 versus HG alone (panel B).

FIG. 4 is an image of the overexpression of SphK in endothelial cells. (A) SphK activity was measured in the transfected BAEC stably overexpressing wild-type SphK (SphK^{WT}), dominant-negative SphK (SphK^{G82D}) or empty vector (Vector) exposed to 5.5 mM (NG) or 22 mM glucose (HG) for 3 days. Data are the means ±SEM of three independent experiments. *p<0.01 versus NG. (B) The immunoblot was probed with anti-FLAG monoclonal antibodies (M2), showing the expression of SphK^{WT} and SphK^{G82D} in the transfected BAEC.

FIG. 5 is an image of the effect of SphK on high glucose-induced leukocyte adhesion to endothelial cells. The transfected BAEC stably overexpressing SphK^{WT}, SphK^{G82D} or empty vector were exposed to 5.5 mM (NG) or 22 mM glucose (HG) for 3 days. (A) Adherence of U937 cells to the treated BAEC was microscopically photographed (20×). (B) The number of U937 cells adhering to BAEC was determined by visually counting 6 microscopic fields per culture well in triplicate (n=12). Data are mean ±S.D. from one experiment and representative of three independent experiments. *p<0.01.

FIG. 6 is a graphical representation of the role of Gi proteins in the SphK-mediated endothelial phenotype. Confluent monolayers of HUVEC were incubated for 3 days with 5.5 mM (NG), 22 mM (HG) glucose or HG plus 50 ng/ml pertussis toxin (HG+PTX). Then, the cell surface adhesion molecule expression (A) and leukocyte adhesion (B) were measured. (C) E-selectin expression was assayed in HUVEC treated with 5 μM S1P, LPA, dihydro-S1P or vehicle alone for 6 hrs in the presence or absence of PTX (50 ng/ml). Data are mean ±SEM (n=3). *p<0.01, †p <0.05 versus cells without PTX treatment.

FIG. 7 is a graphical representation of the effect of PKC and ERK on high glucose-induced SphK activity. HUVEC were exposed to 5.5 mM (NG) or 22 mM glucose (HG) for 3 days. SphK activity was measured after treatment for 30 min with or without GF109203X (GFX, 5 μM), PD98059 (PD9, 10 μM) or U0126 (U01, 2 μM). Data are mean ±SEM (n=3). *p<0.01, †p <0.05 versus HG alone.

FIG. 8 is a graphical representation of the effect of SphK on high glucose-induced NF-κB activation. Confluent monolayers of HUVEC and BAEC overexpressing SphK^{G82D} or empty vector (Vector) were incubated for 3 days with 5.5 mM (NG), 22 mM (HG) glucose or HG plus 2.5 μM DMS (HG +DMS), or treated with 5 μM S1P for 30 min. Then, NF-κB activity was determined by gel shift assay of NF-κB DNA binding complex as described in ‘Methods’. * indicates the specificity of NF-κB binding defined by competition analyses with the addition of a 50-fold molar excess of unlabelled NF-κB oligonucleotides. Data are representative of similar results in at least three separate experiments.

### DETAILED DESCRIPTION OF THE INVENTION

The present invention is predicated, in part, on the determination that hyperglycaemia-induced endothelial cell functioning is mediated by sphingosine kinase signalling. This development now permits the rational design of therapeutic and/or prophylactic methods for treating adverse endothelial cell functional outcomes in the context of disease conditions which are characterised by hyperglycaemia and/or diabetes mellitus, per se.

Accordingly, one aspect of the present invention is directed to a method of modulating hyperglycaemia-induced endothelial cell functioning, said method comprising modulating the functioning of sphingosine kinase mediated signalling in said cell wherein down-regulating said sphingosine kinase signalling down-regulates said endothelial cell activity

Reference to “endothelial cell” should be understood as a reference to the endothelial cells which line the blood vessels, lymphatics or other serous cavities such as fluid filled cavities. The phrase “endothelial cell” should also be understood as a reference to endothelial cell mutants. “Mutants” include, but are not limited to, endothelial cells which have been naturally or non-naturally modified such as cells which are genetically modified.

It should also be understood that the endothelial cells of the present invention may be at any differentiative stage of development. Accordingly, although committed to differentiating along the endothelial cell lineage, the cells may be immature and therefore not fully functional in the absence of further differentiation, such as CD34⁺ progenitor cells. Preferably, the subject endothelial cell is a vascular endothelial cell.

Accordingly, the present invention more preferably provides a method of modulating hyperglycaemia-induced vascular endothelial cell functioning, said method comprising modulating the functioning of sphingosine kinase mediated signalling in said cell wherein down-regulating said sphingosine kinase signalling down-regulates said vascular endothelial cell activity.

Reference to endothelial cell “functioning” should be understood as reference to any one or more of the functional activities which an endothelial cell is capable of performing. This includes, for example, proliferation, differentiation, migration, cell surface molecule expression, sensitization to cytokine stimulation pro-inflammatory cytokine production, neutrophil binding, inflammation and/or angiogenesis. In the context of the present invention, it has been determined that the induction of a hyperglycaemic state results in the up-regulation of sphingosine kinase activity and thereby the onset of adverse endothelial cell functioning, in particular dysfunction of the vascular endothelium such as the onset of diabetic vasculopathy that affects, inter alia, retina, glomeruli, peripheral nerves, cardiovascular tissues, wound healing, and pregnancy. Examples of diabetic vasculopathy include the up-regulation of cell surface adhesion molecules (which can lead to a number of outcomes including inflammation and atherosclerosis) and the subsequent formation of vascular lesions.

Reference to “hyperglycaemia-induced” endothelial cell functioning should be understood as a reference to any one or more endothelial cell functions which are induced by virtue of the onset of a hyperglycaemic state in the extracellular environment of the subject cell. By “hyperglycaemic” is meant a higher glucose concentration than is normally observed in the subject cellular environment. To this end, it should be understood that the subject hyperglycaemic state may be a localised state or a systemic state. To the extent that the hyperglycaemic state is due to diabetes, the hyperglycaemic state is systemic (specifically, an elevated blood glucose level). By “normal” level or “normal” observation for a given cellular environment is meant the level of glucose which occurs in a corresponding cellular environment of an individual who exhibits normal glucose metabolism. In this regard, normal systemic mammalian glucose metabolism is characterised by variation in the glucose level relative to a mammal's ingestion of nutrients. Accordingly, the normal level of glucose in a mammal will correlate to a range of levels which form a sequential cycle relative to insulin secretion. A hyperglycaemic level is a level which falls above the normal range when considered relative to this defined cycle of glucose metabolism. An analogous definition should be understood to apply in respect of localised glucose levels. In general, blood glucose levels (ie systemic levels) are calculated relative to a fasting level of glucose via a glucose tolerance test, this test being well known to the person of skill in the art. This test provides an accurate measure of whether the response of an individual to an ingested glucose load is in fact a hyperglycaemic response. It should also be understood that in some situations it may be preferable that the normal reference level is the level determined from one or more subjects of a relevant cohort to that of the subject being treated by the method of the invention. By “relevant cohort” is meant a cohort characterised by one or more features which are also characteristic of the subject who is the subject of treatment. These features include, but are not limited to, age, gender, ethnicity or health status, for example.

It should be understood that the endothelial cell functional activity which is induced by the hyperglycaemic state may correlate to an entirely aberrant response, such as one which leads to the formation of vascular lesions, or it may be one which in fact correlates to a normal physiological response but is nevertheless unwanted. For example, in many hyperglycaemic episodes one or more aspects of a vascular inflammatory response are observed. In some situations this may in fact correlate to a normal response. However, whether or not such a response is desirable is likely to largely depend on the cause of the hyperglycaemic event. To the extent that the hyperglycaemic event is caused by aberrant insulin production or insulin's action, the subsequent inflammatory response may be physiologically normal but is nevertheless highly undesirable. Accordingly, the present invention provides a means of down-regulating a vascular endothelial functional response which is induced by a hyperglycaemic event but which response is unwanted, irrespective of whether it correlates to a physiologically normal response versus and entirely aberrant and destructive response.

The present invention most preferably provides a method of down-regulating hyperglycaemia-induced vascular endothelial cell functioning, said method comprising modulating the functioning of sphingosine kinase mediated signalling in said cell.

The present study demonstrates that sphingosine kinase is increased in cardiovascular tissues (aorta and heart) from STZ-induced diabetic rats. Moreover, when euglycemia is achieved with insulin treatment in the diabetic rats, the increased sphingosine kinase activity is completely prevented. This reversibility of diabetes-induced increases in sphingosine kinase activity in vasculature following insulin administration not only indicates a previously unknown molecular mechanism underlying the hyperglycaemic damage, but also represents a pharmacotherapeutic target for the protection of vascular lesions in diabetic patients.

Without limiting the present invention to any one theory or mode of action, the mechanism of insulin-induced inhibition of sphingosine kinase in vivo is thought to be related to a reduction in the intra-/extra-cellular glucose concentration. This notion is further characterized by the in vitro studies which show a direct effect of high glucose on sphingosine kinase activation in vascular endothelial cells, whereas insulin itself has no inhibitory effect on the enzyme activity in vitro. Treatment of either HUVEC or BAEC under chronic high glucose conditions results in profound increases in not only sphingosine kinase activity but also production of S1P. This effect of glucose is time dependent with increases in sphingosine kinase activity and S1P production evident after 3-days of treatment. No significant changes in sphingosine kinase activity or S1P production are observed over the short term (minutes to <3-days), suggesting a chronic effect of high glucose. Unlike endothelial cells, aortic smooth muscle cells are able to maintain a normal intracellular glucose concentration that results in no significant change in sphingosine kinase activity under high glucose conditions. Furthermore, non-metabolizable L-glucose or mannitol at 22 mmol/L fails to activate sphingosine kinase or effect S1P production, indicating a specific effect of hyperglycaemia on sphingosine kinase activation in endothelial cells, principally due to the surplus cellular metabolites of D-glucose within the cells.

Accordingly, another aspect of the present invention is directed to a method of down-regulating hyperglycaemia-induced vascular endothelial cell dysfunction, said method comprising down-regulating the functioning of sphingosine kinase mediated signalling in said cell.

Preferably, said vascular endothelial cell dysfunction is the cellular abnormalities in said cells that cause vasculopathy and even more preferably up-regulation of endothelial cell surface adhesion molecule expression, increased endothelial permeability, abnormalities in vascular regeneration, contractility and blood flow, aberrant coagulation, or vascular inflammation.

In a related aspect, it has also been determined that the onset of diabetes, per se, will induce adverse endothelial cell functioning. Although hyperglycaemia, which is characteristic of diabetes, has been determined to induce adverse endothelial cell functioning, it has also been found this is not an exclusive trigger of adverse endothelial cell functioning. Rather, diabetes provides a number of other triggers of adverse endothelial cell functioning and the subsequent vascular complications.

Accordingly, a related aspect of the present invention is directed to a method of modulating diabetes-induced endothelial cell functioning, said method comprising modulating the functioning of sphingosine kinase mediated signalling in said cell wherein downregulating said sphingosine kinase signalling downregulates said endothelial cell activity.

Preferably, said endothelial cell is a vascular endothelial cell.

More preferably, said endothelial cell functioning is downregulated and said adverse endothelial cell functioning are the cellular abnormalities which cause vasculopathy and even more preferably upregulation of cell surface adhesion molecule expression, increased endothelial permeability, abnormalities in vascular regeneration, contractility and blood flow, aberrant coagulation or vascular inflammation.

Reference to “diabetes” should be understood as a reference to a condition in which insufficient levels and activities of insulin are produced to maintain biologically normal glucose levels. As detailed hereinbefore, the diabetes which is the subject of the present invention may either be due to a congenital defect in the pancreatic islet cells, the onset of an autoimmune response directed to the pancreatic β-cells (for example Type 1 diabetes/IDDM, slowly progressive adult onset IDDM which is also referred to as latent autoimmune diabetes in adults or LADA), defects in insulin's action and the functioning of the pancreatic islet cells caused by dietary factors or stress (for example Type 2 diabetes/adult onset diabetes non-insulin dependent diabetes mellitus, NIDDM), damage to the pancreatic islet cells such as, but not limited to, that caused by physical injury, the degeneration of pancreatic islet cells due to any one of a number of non-autoimmune conditions or as a side-effect to the onset or treatment of an unrelated disease condition. Accordingly, “diabetes” as referred to herein includes Type 1 diabetes, Type 2 diabetes and other diabetic conditions including gestational diabetes.

Reference to “sphingosine kinase” should be understood as reference to all forms of this protein and to functional derivatives and homologues thereof. This includes, for example, any isoforms which arise from alternative splicing of the subject sphingosine kinase mRNA or functional mutants or polymorphic variants of these proteins. For example, this definition extends to the isoforms sphingosine kinase-1 and sphingosine kinase-2.

Reference to “sphingosine kinase mediated signalling” should be understood as a reference to the intracellular signalling pathway which utilises one or both of sphingosine kinase and/or sphingosine-1-phosphate or functional derivatives of homologues thereof. Sphingosine kinase is a key regulatory enzyme in the activity of the sphingosine kinase signalling pathway and functions to generate the endogenous sphingolipid mediator sphingosine-1-phosphate. Still further, and without limiting the present invention in any way, sphingosine kinase and sphingosine-1-phosphate are thought to be part of a signalling cascade in which ERK1/2 act to phosphorylate and activate sphingosine kinase. Similarly, PKC is also known to play a role in sphingosine kinase activation, although PKC is nevertheless thought to act via ERK1/2. Ultimately, it is believed that this signalling pathway leads to an increase in the binding activity of NK-κB to the promoter regions of many inflammatory genes.

As detailed hereinbefore, it has been determined that the adverse endothelial cell functional activities which are observed in hyperglycaemic patients are the result of the increased activity of sphingosine kinase which is induced by increased levels of glucose. Accordingly, reference to modulating the “functioning” of sphingosine kinase mediated signalling should be understood as a reference to modulating the level of sphingosine kinase activity which is present in any given cell as opposed to the concentration of sphingosine kinase, per se. Although a decrease in the intracellular concentration of sphingosine kinase will generally correlate to a decrease in the level of sphingosine kinase functional activity which is observed in a cell, the person skilled in the art would also understand that decreases in the level of activity can be achieved by means other than merely decreasing absolute intracellular sphingosine kinase concentrations. For example, one might utilise means of decreasing the half life of sphingosine kinase or sterically hindering the binding of this molecule to its substrate.

It should also be understood that reference to modulation of sphingosine kinase mediated signalling, in particular its down-regulation, does not necessarily mean that the activity of this signalling pathway need be returned to physiologically normal levels. Rather, the level need only be one which is changed relative to the pretreatment level. Accordingly, the method of the present invention may be applied to improve adverse vascular endothelial cell function in some situations while in other situations it may be desirable or necessary to completely normalise vascular endothelial cell functioning. The subject modulation may be transient or long term, depending on the requirements of the particular situation.

Accordingly, modulation of the “activity” of sphingosine kinase mediated signalling should be understood as a reference to either up-regulating or down-regulating the signalling mechanism. Although the preferred method is to down-regulate the subject signalling in the context of a hyperglycaemic patient exhibiting adverse endothelial cell functioning, there may be certain circumstances where it is desirable to up-regulate sphingosine kinase signalling, for example where glucose levels have decreased (e.g. subsequently to insulin treatment) and the method of the invention has led to levels of signalling activity which are perhaps too low. Up-regulation may therefore be necessary in order to normalise sphingosine kinase signalling levels. Either form of modulation may be achieved by any suitable means and include:
(i) modulating absolute levels of the components of the sphingosine kinase mediated signalling pathway, such as sphingosine kinase and/or sphingosine-1-phosphate, such that either more or less of these molecules are available for activation and/or to interact with downstream targets.
(ii) agonising or antagonising the components of the sphingosine kinase mediated signalling pathway, such as sphingosine kinase and/or sphingosine-1-phosphate, such that the functional effectiveness of any one or more of these molecules is either increased or decreased. For example, increasing the half life of sphingosine kinase may achieve an increase in the overall level of sphingosine kinase activity without actually necessitating an increase in the absolute intracellular concentration of sphingosine kinase. Similarly, the partial antagonism of sphingosine kinase or sphingosine-1-phosphate, for example by coupling these molecules to components that introduce some steric hindrance in relation to their binding to downstream targets, may act to reduce, although not necessarily eliminate, the effectiveness of the signalling which they provide. Accordingly, this may provide a means of down-regulating sphingosine kinase mediated signalling without necessarily down-regulating the absolute concentrations of the components of this pathway.

In terms of achieving the up or down-regulation of sphingosine kinase mediated signalling, means for achieving this objective would be well known to the person of skill in the art and include, but are not limited to:
(i) introducing into a cell a nucleic acid molecule encoding a sphingosine kinase signalling pathway component or functional equivalent, derivative or analogue thereof in order to up-regulate the capacity of said cell to express the sphingosine kinase mediated pathway component;
(ii) introducing into a cell a proteinaceous or non-proteinaceous molecule which modulates transcriptional and/or translational regulation of a gene, wherein this gene may be any sphingosine kinase signalling pathway component, in particular sphingosine kinase or sphingosine-1-phosphate or functional portion thereof, or some other gene which directly or indirectly modulates the expression of the components of sphingosine kinase mediated signalling pathways;
(iii) introducing into a cell one or more of the sphingosine kinase mediated signalling pathway component expression products (in either active or inactive form) or a functional derivative, homologue, analogue, equivalent or mimetic thereof;
(iv) introducing a proteinaceous or non-proteinaceous molecule which functions as an antagonist of sphingosine-1-phosphate to occupy and inactivate sphingosine-1-phosphate receptors or an inhibitor of sphingosine-1-phosphate receptors which downregulates sphingosine-1-phosphate receptor functioning;
(v) introducing into a cell a proteinaceous or non-proteinaceous molecule which modulates the expression and/or function of sphingosine-1-phosphate receptors (for example, pertussis toxin);
(vi) introducing a proteinaceous or non-proteinaceous molecule which functions as an antagonist to any one or more components of the sphingosine kinase signalling pathway expression product such as GF109203X (PKC inhibitor), PD98059 (MEK1/2 inhibitor), U0126 (MEK1/2 inhibitor), N′N′-dimethylsphingosine (sphingosine kinase chemical inhibitor) or SphK^{G82D} (mutant sphingosine kinase dominant negative).
(vii) introducing a proteinaceous or non-proteinaceous molecule which functions as an agonist of the sphingosine kinase mediated signalling pathway expression product.
(viii) introducing a proteinaceous or non-proteinaceous molecule which down-regulates or abolishes the ability of glucose to induce activation of the sphingosine kinase signalling pathway.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the sphingosine kinase signalling pathway components or small molecules capable of acting as agonists or antagonists.

Chemical agonists may not necessarily be derived from the components of the sphingosine kinase mediated signalling pathway product but may share certain conformational similarities. Alternatively, chemical agonists may be specifically designed to meet certain physiochemical properties. Antagonists may be any compound capable of blocking, inhibiting or otherwise preventing components of the sphingosine kinase mediated signalling pathway from carrying out their normal biological function, such as molecules which prevent activation or else prevent the downstream functioning of activated molecules. Antagonists include monoclonal antibodies, dominant-negative sphingosine kinase mutants and antisense nucleic acids which prevent transcription or translation of the genes or mRNA of components of the sphingosine kinase mediated signalling pathway in mammalian cells. Modulation of expression may also be achieved utilising antigens, RNA (particularly siRNA), ribosomes, DNAzymes, RNA aptamers, antibodies or molecules suitable for use in cosuppression. The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the sphingosine kinase gene (or any other gene which encodes a component of the sphingosine kinase signalling pathway) or functional equivalent or derivative thereof with an agent and screening for the modulation of sphingosine kinase protein production or functional activity, modulation of the expression of a nucleic acid molecule encoding sphingosine kinase or modulation of the activity or expression of a downstream sphingosine kinase cellular target. Detecting such modulation can be achieved utilising techniques such as Western blotting, electrophoretic mobility shift assays and/or the readout of reporters of sphingosine kinase activity such as luciferases, CAT and the like.

It should be understood that the sphingosine kinase gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate sphingosine kinase activity, at either the nucleic acid or expression product levels, or the gene may require activation—thereby providing a model useful for, inter alia, screening for agents which up regulate sphingosine kinase expression. Further, to the extent that a sphingosine kinase nucleic acid molecule is transfected into a cell, that molecule may comprise the entire sphingosine kinase gene or it may merely comprise a portion of the gene such as the portion which regulates expression of the sphingosine kinase product. For example, the sphingosine kinase promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream sphingosine kinase regulatory target (for example, sphingosine-1-phosphate), rather than sphingosine kinase itself. Yet another example includes sphingosine kinase binding sites ligated to a minimal reporter. For example, modulation of sphingosine kinase activity can be detected by screening for the modulation of the functional activity of an endothelial cell. This is an example of an indirect system where modulation of sphingosine kinase expression, per se, is not the subject of detection. Rather, modulation of the molecules which sphingosine kinase regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the sphingosine kinase nucleic acid molecule or expression product itself or which modulate the expression of an upstream molecule, which upstream molecule subsequently modulates sphingosine kinase expression or expression product activity. Accordingly, these methods provide a mechanism of detecting agents which either directly or indirectly modulate sphingosine kinase expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound or otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of sphingosine kinase or the activity of the sphingosine kinase expression product. Said molecule acts directly if it associates with the sphingosine kinase nucleic acid molecule or expression product to modulate expression or activity, respectively. Said molecule acts indirectly if it associates with a molecule other than the sphingosine kinase nucleic acid molecule or expression product which other molecule either directly or indirectly modulates the expression or activity of the sphingosine kinase nucleic acid molecule or expression product, respectively. Accordingly, the method of the present invention encompasses the regulation of sphingosine kinase nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to up-regulation or down-regulation.

“Derivatives” of the molecules herein described (for example sphingosine kinase, sphingosine-1-phosphate or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, sphingosine kinase or derivative thereof may be fused to a molecule to facilitate its entry into a cell. Analogs of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” of sphingosine kinase or sphingosine-1-phosphate should be understood to mean molecules which exhibit at least some of the functional activity of the form of sphingosine kinase or sphingosine-1-phosphate of which it is a variant. A variation may take any form and may be naturally or non-naturally occurring. A mutant molecule is one which exhibits modified functional activity.

By “homologue” is meant that the molecule is derived from a species other than that which is being treated in accordance with the method of the present invention. This may occur, for example, where it is determined that a species other than that which is being treated produces a form of sphingosine kinase or sphingosine-1-phosphate which exhibits similar and suitable functional characteristics to that of the sphingosine kinase or sphingosine-1-phosphate which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing sphingosine kinase and/or sphingosine-1-phosphate analogues which exhibit properties such as more potent pharmacological effects. Sphingosine kinase and/or sphingosine-1-phosphate or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances. In addition to screening for molecules which mimic the activity of sphingosine kinase and/or sphingosine-1-phosphate, it may also be desirable to identify and utilise molecules which function agonistically or, most preferably, antagonistically to sphingosine kinase and/or sphingosine-1-phosphate in order to up or down-regulate the functional activity of sphingosine kinase and/or sphingosine-1-phosphate in relation to modulating smooth muscle cell activity. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of sphingosine kinase and/or sphingosine-1-phosphate capable of acting as agonists or antagonists. Chemical agonists may not necessarily be derived from sphingosine kinase and/or sphingosine-1-phosphate but may share certain conformational similarities. Alternatively, chemical agonists may be specifically designed to mimic certain physiochemical properties of sphingosine kinase and/or sphingosine-1-phosphate. Antagonists may be any compound capable of blocking, inhibiting or otherwise preventing sphingosine kinase and/or sphingosine-1-phosphate from carrying out its normal biological functions. Antagonists include monoclonal antibodies specific for sphingosine kinase and/or sphingosine-1-phosphate or parts of sphingosine kinase and/or sphingosine-1-phosphate.

Analogues of sphingosine kinase and/or sphingosine-1-phosphate or of sphingosine kinase and/or sphingosine-1-phosphate agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with dimethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | | |
| 1-carboxy-1-(2,2-diphenyl- | Nmbc | carbamylmethyl)glycine | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The method of the present invention contemplates the modulation of hyperglycaemia-induced endothelial cell functioning both in vitro and in vivo. Although the preferred method is to treat an individual in vivo it should nevertheless be understood that it may be desirable that the method of the invention may be applied in an in vitro environment, for example to provide an in vitro model for the analysis of vascular aberrancies such as the formation of vascular lesions or other such symptom which may have relevance to a disease condition other than just hyperglycaemia-related conditions. In another example the application of the method of the present invention to an in vitro environment may extend to providing a readout mechanism for screening technologies such as those hereinbefore described. That is, molecules identified utilising these screening techniques can be assayed to observe the extent and/or nature of their functional effect on hyperglycaemia-induced endothelial cell functioning.

Although the preferred method is to down-regulate, hyperglycaemia-mediated endothelial cell functioning (for example in order to down-regulate the progression of diabetes-related vascular diseases), it should be understood that there may also be circumstances in which it is desirable to up-regulate the subject functional activity (for example in order to upregulate vascular regeneration during wound healing and rescue of myocardial infarction).

In a related aspect the present invention is directed to a method of modulating hyperglycaemia-induced endothelial cell functioning in a mammal, said method comprising modulating the functioning of sphingosine kinase mediated signalling in said mammal wherein down-regulating sphingosine kinase signalling down-regulates said endothelial cell activity.

More particularly, the present invention provides a method of modulating hyperglycaemia-induced vascular endothelial cell functioning in a mammal, said method comprising modulating the functioning of sphingosine kinase mediated signalling in said mammal wherein down-regulating sphingosine kinase signalling down-regulates said vascular endothelial cell activity.

Preferably, said vascular endothelial cell functioning is vascular endothelial cell dysfunction and said modulation of functional activity is the down-regulation of said activity.

More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably up-regulation of endothelial cell surface adhesion molecule expression, vascular inflammation or atherogenic lesions.

Most preferably, the present invention provides a method of down-regulating hyperglycaemia-induced vascular endothelial cell functioning in a mammal, said method comprising down-regulating the functioning of sphingosine kinase mediated signalling in said mammal.

In accordance with this preferred embodiment, said vascular endothelial cell functioning is preferably vascular endothelial cell dysfunction. More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably up-regulation of endothelial cell surface adhesion molecule expression, vascular inflammation or atherogenic lesions.

In a most preferred embodiment, said down-regulation of sphingosine kinase mediated signalling is achieved by administering GF109203X, PD98059, U0126, N′N′-dimethylsphingosine or SphK^{G82D}.

In another related aspect the present invention is directed to a method of modulating diabetes-induced endothelial cell functioning in a mammal, said method comprising modulating the functioning of sphingosine kinase mediated signalling in said mammal wherein downregulating sphingosine kinase signalling downregulates said endothelial cell activity.

More particularly, the present invention provides a method of modulating diabetes induced vascular endothelial cell functioning in a mammal, said method comprising modulating the functioning of sphingosine kinase mediated signalling in said mammal wherein downregulating sphingosine kinase signalling downregulates said vascular endothelial cell activity.

Preferably, said vascular endothelial cell functioning is vascular endothelial cell dysfunction and said modulation of functional activity is the downregulation of said activity.

More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably upregulation of endothelial cell surface adhesion molecule expression, vascular inflammation or atherogenic lesions.

Most preferably, the present invention provides a method of downregulating diabetes-induced vascular endothelial cell functioning of sphingosine kinase mediated signalling in said mammal.

In a most preferred embodiment, said downregulation of sphingosine kinase mediated signalling is achieved by administering GF109203X, PD98059, U0126, N′N′-dimethylsphingosine or SphKG^{G82D}.

A further aspect of the present invention relates to the use of the invention in relation to the treatment and/or prophylaxis of disease conditions. Without limiting the present invention to any one theory or mode of action, the ever growing epidemic of diabetes in Western society renders hyperglycaemia-induced vascular endothelial cell dysfunction a serious problem, the regulation of which is likely to become an integral component of the management of such diseases. Accordingly, the method of the present invention provides a valuable tool for modulating aberrant or otherwise unwanted endothelial cell functioning which has been induced by virtue of the onset of a hyperglycaemic state. To this end, it should be understood that to the extent that this aspect of the present invention discusses the treatment of a “condition characterised by hyperglycaemia-mediated vascular endothelial cell induced functioning”, the present invention is also directed to treating the unwanted vascular endothelial cell functioning that is a symptom of some hyperglycaemic conditions, such as diabetes, rather than the hyperglycaemia itself.

Accordingly, yet another aspect of the present invention is directed to a method for the treatment and/or prophylaxis of a condition in a mammal, which condition is characterised by aberrant, unwanted or otherwise inappropriate hyperglycaemia-induced endothelial cell functioning, said method comprising modulating the functional activity of sphingosine kinase mediated signalling in said cell wherein down-regulating sphingosine kinase signalling down-regulates said endothelial cell activity.

More particularly, the present invention is directed to a method for the treatment and/or prophylaxis of a condition in a mammal, which condition is characterised by aberrant, unwanted or otherwise inappropriate hyperglycaemia-induced vascular endothelial cell functioning, said method comprising modulating the functional activity of sphingosine kinase mediated signalling in said cell wherein down-regulating sphingosine kinase signalling down-regulates said vascular endothelial cell activity.

Preferably, said vascular endothelial cell functioning is vascular endothelial cell dysfunction and said modulation of functional activity is the down-regulation of said activity.

More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably up-regulation of endothelial cell surface adhesion molecule expression, vascular inflammation or atherogenic lesions.

Most preferably, the present invention is directed to a method for the treatment and/or prophylaxis of a condition in a mammal, which condition is characterised by aberrant, unwanted or otherwise inappropriate hyperglycaemia-induced vascular endothelial cell functioning, said method comprising down-regulating the functional activity of sphingosine kinase mediated signalling in said cell.

In accordance with this preferred embodiment, said vascular endothelial cell functioning is preferably vascular endothelial cell dysfunction. More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably up-regulation of endothelial cell surface adhesion molecule expression, vascular inflammation or atherogenic lesions.

Still more preferably, said condition is type 1 or type 2 diabetes, Cushing's disease, Cusing's syndrome, hyperthyroidism, metabolic syndrome or acromegalic.

In a most preferred embodiment, said down-regulation of sphingosine kinase mediated signalling is achieved by administering GF109203X, PD98059, U0126, N′N′-dimethylsphingosine or SphK^{G82D}.

The present invention therefore most particularly provides a method for the treatment and/or prophylaxis of a symptom of diabetes, which symptom is characterised by aberrant, unwanted or otherwise inappropriate vascular endothelial cell functioning, said method comprising down-regulating the functional activity of said sphingosine kinase mediated signalling in said cell.

Preferably, said vascular endothelial cell functioning is vascular endothelial cell dysfunction and said modulation of functional activity is the down-regulation of said activity.

More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably up-regulation of endothelial cell surface adhesion molecule expression, vascular inflammation or atherogenic lesions.

Most preferably, said symptom is diabetes-related vascular diseases involving retina, kidney, peripheral nerves and atherosclerosis.

Still more preferably, said symptom is induced by hyperglycaemia.

In a still more preferred embodiment, said down-regulation of sphingosine kinase mediated signalling is achieved by administering GF109203X, PD98059, U0126, pertussis toxin, N′N′-dimethylsphingosine or SphK^{G82D}.

The most preferred embodiments of this aspect of the present invention preferably facilitate the subject endothelial cell functioning being reduced, retarded or otherwise inhibited. Reference to “reduced, retarded or otherwise inhibited” should be understood as a reference to inducing or facilitating the partial or complete inhibition of said functioning.

The subject of the treatment or prophylaxis is generally a mammal such as but not limited to human, primate, livestock animal (eg. sheep, cow, horse, donkey, pig), companion animal (eg. dog, cat), laboratory test animal (eg. mouse, rabbit, rat, guinea pig, hamster), captive wild animal (eg. fox, deer). Preferably the mammal is a human or primate. Most preferably the mammal is a human.

An “effective amount” means an amount necessary to at least partly attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the agent together with subjection of the mammal to other agents, drugs or treatments which may be useful in relation to the treatment of the subject condition such as insulin administration in the context of diabetes.

Administration of the modulatory agent, in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention relates to the use of an agent capable of modulating sphingosine kinase mediated signalling in the manufacture of a medicament for the regulation of hyperglycaemia-induced endothelial cell functioning in a mammal wherein down-regulating sphingosine kinase signalling down-regulates said endothelial cell activity.

More particularly, the present invention relates to the use of an agent capable of modulating sphingosine kinase mediated signalling in the manufacture of a medicament for the regulation of hyperglycaemia-induced vascular endothelial cell functioning in a mammal wherein down-regulating sphingosine kinase signalling down-regulates said vascular endothelial cell activity.

Preferably, said vascular endothelial cell functioning is vascular endothelial cell dysfunction and said modulation of functional activity is the down-regulation of said activity.

More preferably, said vascular endothelial cell dysfunction is vasculopathy including both microvasculopathy that includes lesions in microvascular beds of the retina, renal glomeruli or nerve tissue, and macrovasculopathy that includes lesions in the coronary or peripheral large blood vessels, and even more preferably up-regulation of endothelial cell surface adhesion molecule expression, vascular inflammation or atherogenic lesions.

Most particularly, the present invention relates to the use of an agent capable of down-regulating sphingosine kinase mediated signalling in the manufacture of a medicament for the regulation of hyperglycaemia-induced vascular endothelial cell functioning in a mammal.

Most preferably, said condition is diabetes.

In a most preferred embodiment, said down-regulation of sphingosine kinase mediated signalling is achieved by administering GF109203X, PD98059, U0126, pertussis toxin, N′N′-dimethylsphingosine or SphKG^{G82D}.

In yet another further aspect, the present invention contemplates a pharmaceutical composition comprising the modulatory agent as hereinbefore defined together with one or more pharmaceutically acceptable carriers and/or diluents. These agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Various methods of transferring or delivering DNA to cells for expression of the gene product protein, otherwise referred to as gene therapy, are disclosed in Gene Transfer into Mammalian Somatic Cells in vivo, N. Yang, Grit. Rev. Biotech. 12(4):335-356 (1992), which is hereby incorporated by reference.

Strategies for treating these medical problems with gene therapy include therapeutic strategies such as identifying a defective gene or protein and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that for a sphingosine kinase antagonist may be placed in a patient and thus prevent or mitigate the occurrence of adverse hyperglycaemia induced endothelial cell functioning.

Many protocols for transfer of genetic regulatory sequences are envisioned in this invention. Transfection of promoter sequences, or other sequences which would modulate the expression and/or activity of sphingosine kinase or other related signalling molecule are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate the subject gene.

Gene transfer methods for gene therapy fall into three broad categories: physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer.

Chemical methods of gene therapy may involve a lipid based compound, not necessarily a liposome, to ferry the DNA across the cell membrane. Lipofectins or cytofectins, lipid-based positive ions that bind to negatively charged DNA, may be used to cross the cell membrane and provide the DNA into the interior of the cell. Another chemical method may include receptor-based endocytosis, which involves binding a specific ligand to a cell surface receptor and enveloping and transporting it across the cell membrane.

Many gene therapy methodologies employ viral vectors such as retrovirus vectors to insert genes into cells. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors may be selected from the group including, but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors and are preferred. Adenoviral vectors may be delivered bound to an antibody that is in turn bound to collagen coated stents.

Mechanical methods of DNA delivery may be employed and include, but are not limited to, fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun”, inorganic chemical approaches such as calcium phosphate transfection and plasmid DNA incorporated into polymer coated stents. Ligand-mediated gene therapy, may also be employed involving complexing the DNA with specific ligands to form ligand-DNA conjugates, to direct the DNA to a specific cell or tissue.

The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Gene regulation of sphingosine kinase mediated signalling may be accomplished by administering compounds that bind the sphingosine kinase gene, for example, or control regions associated with the sphingosine kinase gene, or corresponding RNA transcript to modify the rate of transcription or translation. Additionally, cells transfected with a DNA sequence encoding a sphingosine antagonist or agonist may be administered to a patient to provide an in vivo source of a sphingosine kinase regulator. For example, cells may be transfected with a vector containing a nucleic acid sequence encoding a sphingosine kinase signalling pathway regulator.

The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. DNA sequence is operatively linked to an expression control sequence to form an expression vector capable of gene regulation. The transfected cells may be cells derived from the patient's normal tissue, the patient's diseased tissue (such as diseased vascular tissue), or may be non-patient cells. For example, blood vessel cells removed from a patient can be transfected with a vector capable of expressing a regulatory molecule of the present invention, and be re-introduced into the patient. Patients may be human or non-human animals. Cells may also be transfected by non-vector, or physical or chemical methods known in the art such as electroporation, incorporation, or via a “gene gun”. Additionally, DNA may be directly injected, without the aid of a carrier, into a patient.

The gene therapy protocol for transfecting a regulatory molecule into a patient may either be through integration of the regulatory molecule's DNA into the genome of the cells, into minichromosomes or as a separate replicating or non-replicating DNA construct in the cytoplasm or nucleoplasm of the cell. Modulation of gene expression and/or activity may continue for a long period of time or may be reinjected periodically to maintain a desired level of gene expression and/or activity in the cell, the tissue or organ.

The modulated cells may replace existing cells such that the existing biological functioning of the cells is modulated. Alternatively, the modulated cells may be used to infiltrate existing regions of disease to halt progression of the disease. The replaced cells may be tissue specific for the condition to be treated. They may also be stem cells, which can be induced to differentiate along a specific lineage.

Yet another aspect of the present invention relates to the agent as hereinbefore defined, when used in the method of the present invention.

Another aspect of the present invention provides a method for detecting an agent capable of modulating sphingosine kinase mediated signalling said method comprising contacting a cell or extract thereof containing said sphingosine kinase or its functional equivalent or derivative with a putative agent and detecting an altered expression phenotype associated with endothelial cell functioning.

Reference to “sphingosine kinase” should be understood as a reference to either sphingosine kinase expression product or to a portion or fragment of sphingosine kinase such as the cell membrane localisation. In this regard, the sphingosine kinase expression product is expressed in a cell. The cell may be a host cell which has been transfected with the sphingosine kinase nucleic acid molecule or it may be a cell which naturally contains the sphingosine kinase gene. Reference to “extract thereof” should be understood as a reference to a cell free transcription system.

Reference to detecting an “altered expression phenotype associated with endothelial cell functioning” should be understood as the detection of functional cellular changes associated with modulation of sphingosine kinase signalling. These may be detectable, for example, as intracellular changes or changes observable extracellularly, such as changes in adhesion molecule expression.

Still another aspect of the present invention is directed to agents identified in accordance with the screening method defined herein and to said agents for use in the methods of the present invention.

The present invention is described by reference to the following non-limiting examples.

### EXAMPLE 1

### Activation of the Sphingosine Kinase Signalling Pathway by High-Glucose Mediates the Pro-Inflammatory Phenotype of Endothelial Cells

### Methods

### Animals

Male Sprague-Dawley rats weighing 270-290 g were housed in a room under controlled temperature conditions (22° C.) and 12/12-hr of light/dark cycles. Diabetes was induced with 80 mg/kg streptozotocin (STZ) (Sigma-Aldrich) dissolved in citrate buffer (20 mM, pH 4.5) that was administered as a single intraperitoneal injection. Control rats were injected with an equivalent volume of the vehicle only. After injection (24 h), diabetes was diagnosed by the development of hyperglycaemia (>14 mmol/L blood glucose). One-half of the diabetic rats were randomly selected to receive insulin treatment (Linplant, one implant/200 g body wt; LinShin, Canada). Blood glucose levels were monitored every 4 days using Glucostix reagent strips (Boehringer Mannheim, Indianapolis, Ind.). Two weeks after the onset of diabetes, rats were killed for the study. All experiments were conducted in accordance with the PC-2 procedure of Institute of Medical and Veterinary Science (IMVS) and approved by the IMVS Animal Ethics Committee.

### Cell Culture

Human umbilical vein EC (HUVEC) and bovine aortic EC (BAEC) were routinely cultured in this laboratory as previously described (Verrier et al., 2004, Circ. Res. 94:1515-1522). In all experiments EC ranging from passage 2-6 were used. For the experimental studies, EC were allowed to reach confluence in the regular growth media. Medium was then changed to (i) EBM (Clonetics, Walkersville, Md.) containing 1% FCS and 5.5 mM glucose (normal glucose, NG); (ii) NG medium supplemented with additional glucose to final concentration of 22 mM (high glucose); or (iii) NG medium containing 16.5 mM L-glucose or mannitol that served as an osmotic control for the high glucose medium.

### Plasmids and Transfection

FLAG-tagged human wild-type SphK1 cDNA and the dominant negative SphK1^{G82D} were sub-cloned into pcDNA3 plasmids (Invitrogen, Melbourne, Australia) as previous described (Pitson et al., 2000, J. Biol. Chem. 275:33945-33950). Lipofectamine 2000 (Invitrogen) mediated transfection was performed in BAEC according to the manufacturer's protocols. For stable expression, the transfectants were selected in medium containing 800 μg/ml G418 (Invitrogen). The resulting non-clonal pools of G418-resistant transfected cells were then collected and used to avoid clonal variability. The expression of FLAG-tagged transgenes was determined by Westernblot assay with the antibodies against FLAG (M2, Sigma, Clayton, Australia).

### Assays of SphK Activity and S1P Formation

As described previously (Xia et al., 1999, J. Biol. Chem. 274:34499-34505), SphK activity was routinely determined using D-erythro-sphingosine (Biomol, Plymouth Meeting, Pa.) and [γ³²P]ATP (Geneworks, Adelaide, Australia) as substrates, and defined as picomoles of S1P formed per min per mg protein. The formation of S1P in vivo was measured in the permeabilized cells as previously described (Xia et al., 1998, Proc. Natl. Acad. Sci. U.S.A 95:14196-14201).

PKC Activity Assay

PKC activity was measured in situ as described previously (Xia et al., 1996, J. Clin. Invest 98:2018-2026). Briefly, cells were seeded in 24-well plates and exposed to NG or HG for 3 days. After the indicated treatments, total PKC activity was then determined in permeabilized cells in the presence of 10 μM [γ³²P]ATP (5000 cpm/pmol) and the PKC-specific peptide substrate (RKRTLRRL, 200 μM). The activity was then quantified by scintillation counting and normalized to total protein levels.

### Flow Cytometry Analysis

After the indicated treatment, HUVECs were incubated with primary monoclonal antibodies to VCAM-1, ICAM-1 and E-selectin or an isotype-matched non-relevant antibody for 30 min. Cells were then incubated with FITC-conjugated secondary antibody and fixed in 2.5% formaldehyde. The expression of cell-surface adhesion molecules was measured as fluorescence intensity by use of a Coulter Epics Profile XL flow cytometer, as described previously (Verrier et al., 2004, supra).

### Adherence of U937 Cells to EC

U937 cells (CRL 1593.2; ATCC) were grown in RPMI-1640 medium (GIBCO BRL) containing 10% FCS, collected by low-speed centrifugation and resuspended at a density of 2×10⁵ cells/ml in medium without FCS. EC were seeded into 24-well plates and cultured with NG or HG medium for 3 days after confluence. After washing twice with warm RPMI-1640 medium, the U937 cell suspension (100 μl/well) was added and incubated for 30 minutes at 37° C. Non-adherent cells were removed by rinsing the plates three times with PBS, and the number of adherent cells was then counted under microscopy with at least 6 fields per well culture being quantified.

### Electrophoretic Mobility Shift Assay

Nuclear extracts were prepared from EC exposed to NG or HG with or without the indicated treatment. The double-stranded oligonucleotides used as probes in the experiments included 5′-GGATGCCATTGGGGATTTCCTCTTTACTGGATGT-3′ (SEQ ID NO: 1) which contains a consensus NF-κB binding site in E-selectin promoter (underlined). Gel mobility shift of a consensus NF-κB oligonucleotide was performed by incubating a ³²P-labelled NF-κB probe with 4 μg of nuclear proteins as described previously (Xia et al., 1998, supra). The specific DNA-protein complexes were completely abolished by addition of a 50-fold molar excess of unlabelled E-selectin NF-κB oligonucleotides.

### Statistical Analysis

Data are expressed as mean ±SEM, and n indicates number of experiments. Unpaired Student's t-tests were used for comparison between two groups. For multiple comparisons, results were analyzed by ANOVA followed by the Dunnet's test. A value of p<0.05 was considered statistically significant.

### Results

### Effect of Hyperglycaemia on SphK Activity in STZ-Induced Diabetic Rats.

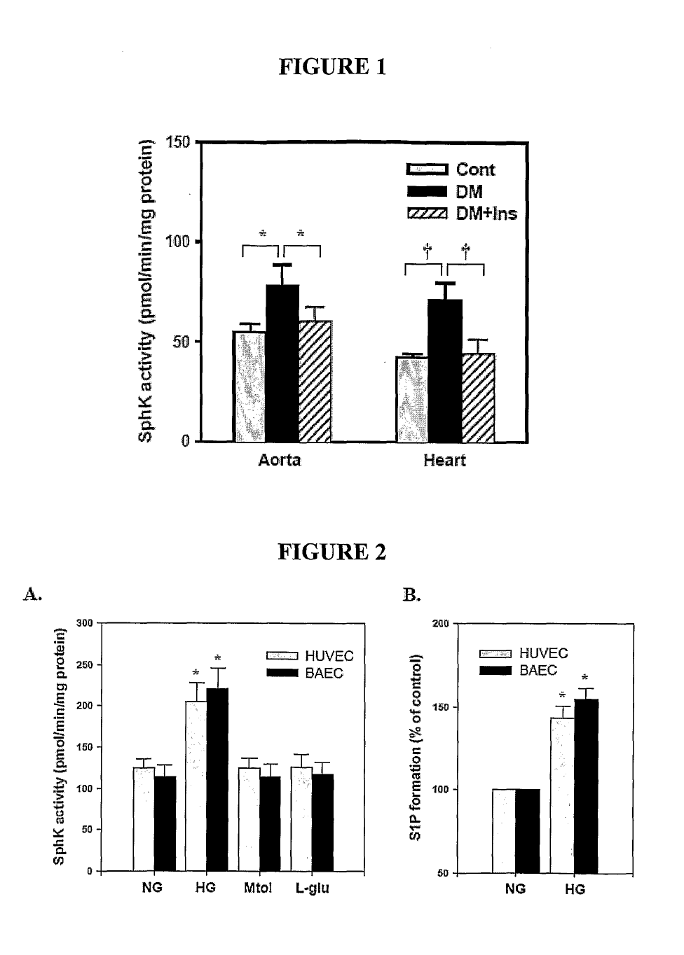

To test our hypothesis that SphK could be an important player in mediating hyperglycaemic damage on vasculature, we firstly examined the extent of SphK activity in vascular tissues from STZ-induced diabetic rats. In those animals demonstrating frank diabetes and a consistent hyperglycaemia (Table 1), total SphK activity in aorta and heart was measured 2 wk post-onset of diabetes. As shown in FIG. 1, SphK activity was significantly increased by 42% (p<0.05) in the aorta and 68% (p<0.01) in the heart of diabetic rats, compared to samples taken from control animals. The institution of glycemic control with the use of an insulin pump achieved near euglycemia within several hours that correlated with a significant reduction in SphK activity in both the aorta and heart from diabetic rats (FIG. 1). Taken together, these data suggested that hyperglycaemia is likely to be a key factor accounting for the increased SphK activity in the vascular tissues in diabetic animals.

### Effects of High Glucose on SphK Activity in EC

To confirm the potential effect of hyperglycaemia on SphK activity and to examine which type of vascular cells are prone to SphK activation under high glucose conditions, established cell culture models were used. HUVEC cultured in high glucose (22 mmol/L) media for 3 days resulted in a 60% increase in SphK activity compared with the cells cultured under normal glucose conditions (p<0.01) (FIG. 2A). Consistent with the increases in enzyme activity, intracellular S1P production was increased by 40% in HUVEC exposed to high glucose for 3 days (FIG. 2B). However, there was no significant change in SphK activity when cells were exposed to high glucose for less than 48 hrs (data not shown), indicating a chronic exposure to high glucose is required for SphK activation in HUVEC. After a 3-day incubation with high glucose, SphK activity was also increased in BAEC by 1.7-fold (FIGS. 2A and 2B), whereas no increased activity was detected in aortic smooth muscle cells (data not shown), indicating an endothelial cell-specific effect of high glucose on SphK. Serving as a control, neither mannitol nor L-glucose at 22 mmol/L had any significant effects on SphK activity in HUVEC or BAEC, ruling out a possible influence of osmotic stress.

### High Glucose-Induced SphK Activity Mediates Endothelial Cell Activation.

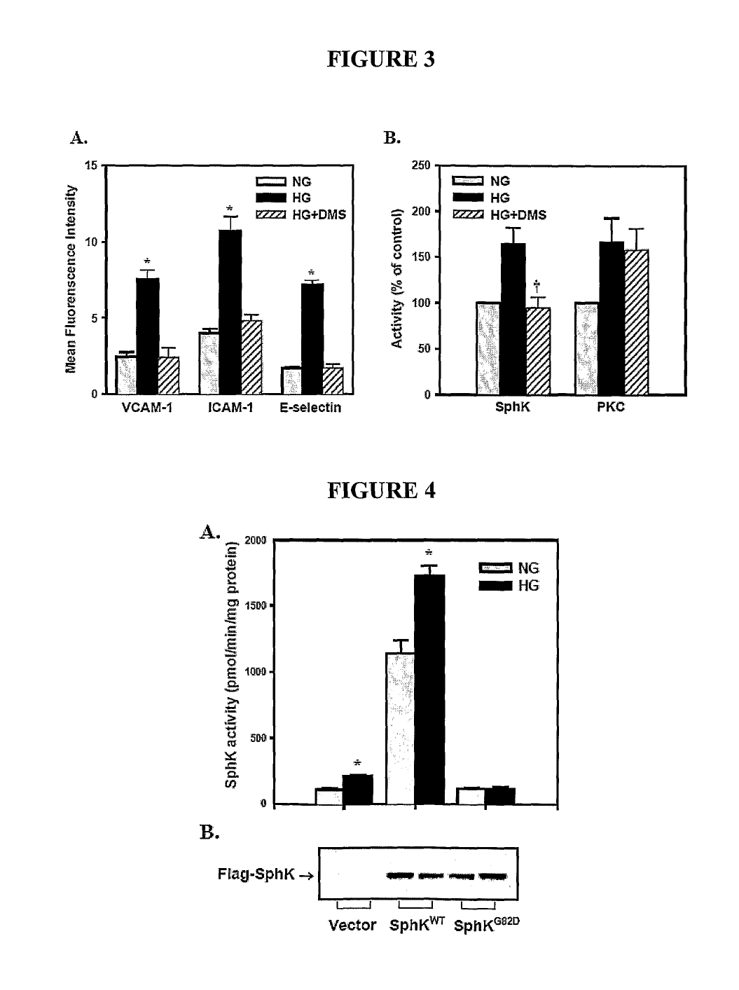

Given the effect of high glucose on SphK activity in endothelial cells, we sought to determine the functional consequences of SphK activation induced by high glucose. In agreement with our previous report (Verrier et al., 2004, supra), exposure of HUVEC to high glucose for 3 days resulted in significant increases in the cell surface expression of VCAM-1, ICAM-1 and E-selectin by 3.1-, 2.7- and 4.2-fold, respectively (FIG. 3A). Interestingly, high glucose-induced increases in VCAM-1, ICAM-1 and E-selectin expression were completely abolished in the presence of N′N′-dimethylsphingosine (DMS), a specific inhibitor of SphK, at a concentration of 2.5 μmol/L (FIG. 3A). At this low concentration, DMS was capable of inhibiting high glucose-induced SphK activity, whereas no inhibitory effect on PKC activity was detected (FIG. 3B), which concurs with the previous report showing the specificity of inhibition by DMS (Edsall et al., 1998, Biochemistry 37:12892-12898). Collectively, these results suggest a critical involvement of SphK activity in endothelial cell activation that results from chronic high exposure.

### SphK Activation is Required for the High Glucose-Induced Pro-Inflammatory Phenotype of Endothelial Cell

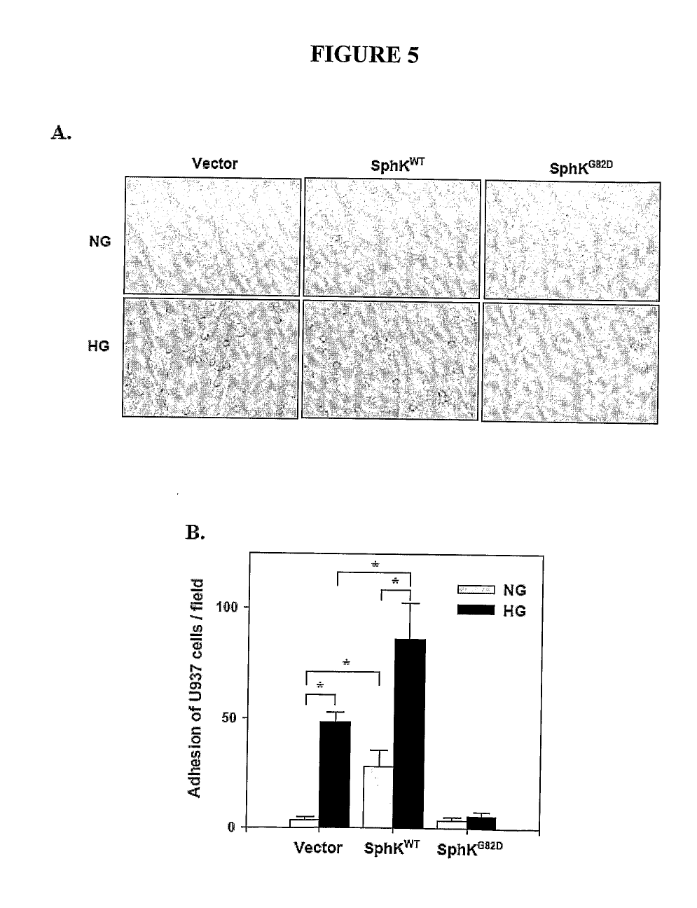

To further verify the role of SphK in mediating high glucose-induced endothelial cell activation, we conducted experiments using a set of genetic approaches. BAEC were stably transfected with constitutively expressing FLAG-tagged wild-type human SphK1 (SphK^{WT}), a major isoform of SphK in endothelial cells (Pitson et al., 2000, Biochem. J. 350 Pt 2:429-441), or a point mutation of SphK1, SphK^{G82D}. SphK^{G82D} has previously been characterized as a dominant-negative mutant that not only lacks the enzymatic activity but also blocks SphK activation in response to any stimuli so far tested (Pitson et al., 2000, supra). Pooled stable transfectants were used to avoid the phenotypic artifacts that may be due to the selection and propagation of individual clones from single transfected cells. Despite SphK^{WT}-transfected BAEC having a 10-fold higher basal level of SphK activity (FIG. 4A), cells cultured in high glucose conditions resulted in a further increase in SphK activity to a similar extent (˜70%) to that seen in the parental cells or the cells transfected with empty vector alone, indicating that the transgenes of SphK are functionally expressed in BAEC and are readily activated in response to high glucose. In contrast, no SphK activation was observed in the cells expressing SphK^{G82D} under high glucose conditions (FIG. 4A), confirming the dominant-negative role of SphK^{G82D} in the transfected BAEC. Given the possible involvement of SphK in high glucose-induced adhesion molecule expression as reported above, we then examined the interaction of endothelial cells with leukocytes in order to testify a pathophysiological relevance of the phenomenon. In agreement with our previous report (Verrier et al. 2004, supra), BAEC exposed to chronic high glucose conditions resulted in a significant increase in the adherence of leukocytes to endothelial cells (FIG. 5). Interestingly, the number of leukocytes adhering to high glucose-stimulated BAEC was markedly enhanced by overexpression of SphK^{WT}, whereas it was profoundly attenuated in the cells expressing SphK^{G82D} (FIG. 5), further supporting a role for SphK activation in mediating the high glucose-induced endothelial cell pro-inflammatory phenotype.

### Effect of S1P Receptors on the SphK-Mediated Endothelial Cell Activation

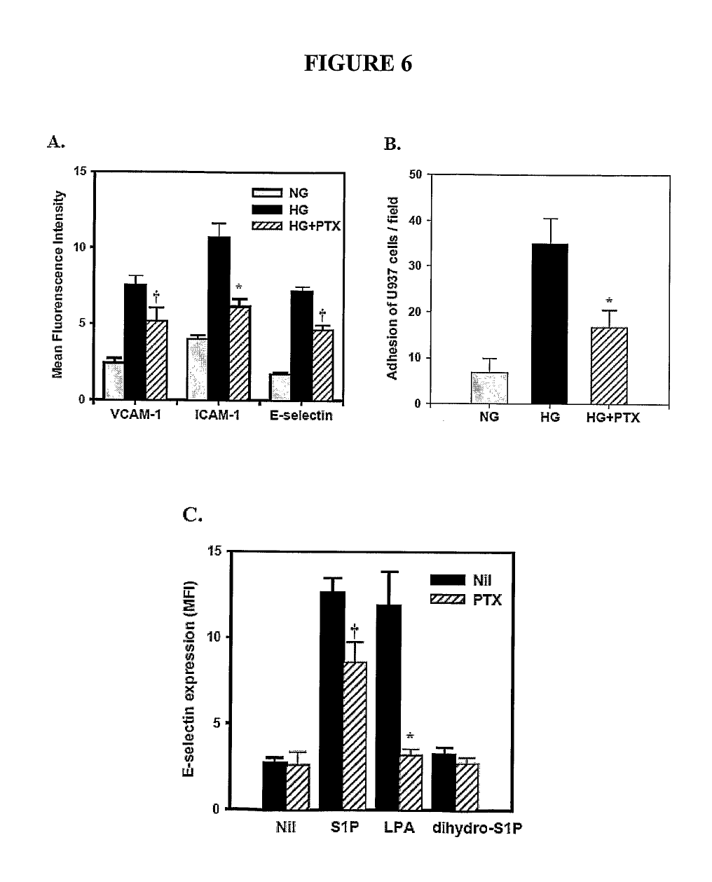

The biological consequences of SphK activation rely on the production of S1P that functions either extracellularly (ie, through S1P receptors) or intracellularly. In order to verify whether S1P receptors are involved in the SphK-mediated endothelial cell activation induced by high glucose, we used pertussis toxin, an inhibitor of Gi proteins, which has previously been shown to block the majority of S1P receptors expressed in endothelial cells (Sanchez et al., 2004. J. Cell Biochem. 92:913-922). As shown in FIG. 6A, treatment of HUVEC with pertussis toxin resulted in only partial inhibition of the high glucose-induced expression of VCAM-1, ICAM-1 and E-selectin by 30%, 42% and 35%, respectively, in comparison with the untreated cells. Moreover, the number of leukocytes adhering to the high glucose-stimulated EC was also partially, but significantly, reduced in HUVEC treated with pertussis toxin (FIG. 6B). These results suggest only a partial involvement of G protein-coupled S1P receptors in the SphK-mediated endothelial cell activation induced by high glucose. Investigating this hypothesis further, HUVEC were then treated with either S1P, lysophosphatidic acid (LPA) or dihydro-S1P (sphinganine-1-phosphate), a S1P analogue that has been shown to specifically bind to S1P receptors with a high affinity, but has no significant intracellular effects (Van Brocklyn et al., 1998, J Cell Biol 142:229-240). Treatment of HUVEC with S1P and LPA resulted in a significant increase in E-selectin expression to similar extents (FIG. 6C). In contrast, dihydro-S1P has no significant effect on E-selectin expression (FIG. 6C). Interestingly, pertussis toxin treatment completely inhibited LPA-induced E-selectin expression, whereas it only partially inhibited the effect of S1P (32%) (FIG. 6C). Taken together, these results suggest that both intra- and extra-cellular effects of S1P are involved in the SphK-dependent endothelial cell activation induced by HG.

### PKC and ERK1/2 Mediate the High Glucose-Induced SphK Activation

The ability of high glucose to activate PKC via de novo synthesis of DAG in endothelial cell has been well documented (Xia et al., 1994, Diabetes 43:1122-1129). Previous studies have suggested a role for PKC in mediating SphK activation and S1P production in HEK 293 cells (Johnson et al., 2002, J. Biol. Chem. 277:35257-35262). We therefore examined a potential role for PKC in the high glucose-induced SphK activation in endothelial cells. Interestingly, treatment of HUVEC with a PKC-specific inhibitor, GF109203X, resulted in an attenuation of the high glucose-induced increases in SphK activity (˜50%; FIG. 7). More recently, we have demonstrated that ERK1/2 were capable of directly activating SphK via the enzyme phosphorylation (Pitson et al., 2003, EMBO J. 22:5491-5500). Using specific inhibitors of the ERK1/2 signalling pathway, either PD98059 or U0126, high glucose-induced increases in SphK activity were completely prevented (FIG. 7). Collectively, these results suggest that ERK1/2, but also PKC to a lesser degree, play important roles in the activation of SphK observed in endothelial cells exposed to high glucose.

### High Glucose Induced NF-κB Activation Dependent on SphK Activity

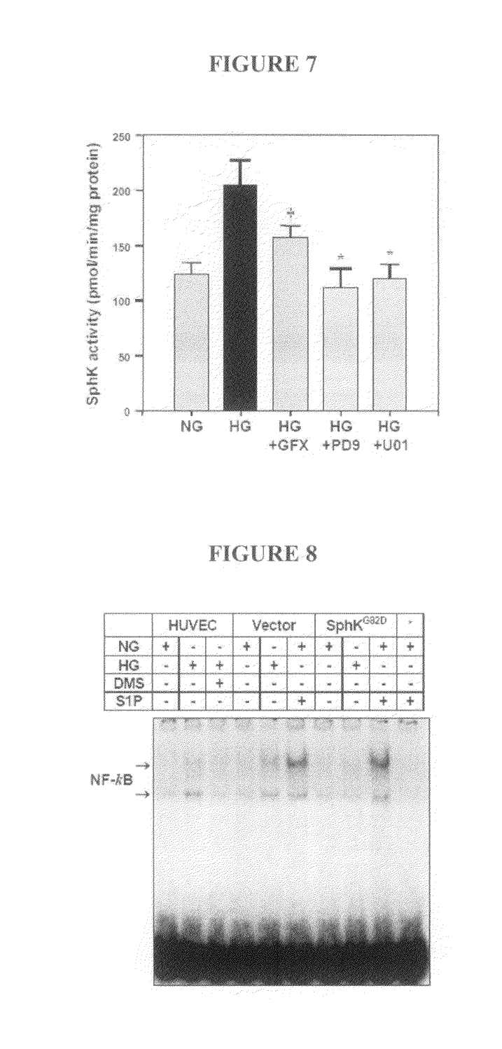

High glucose has been shown to activate the transcription factor NF-κB (Morigi et al., 1998, J. Clin. Invest 101: 1905-1915; Pieper et al., 1997, J. Cardiovasc. Pharmacol. 30:528-532) that is a key transcriptional regulator of a number of pro-inflammatory genes, including adhesion molecules (Read et al., 1994, J. Exp. Med. 179:503-512). We have previously demonstrated that S1P was capable of activating NF-κB and that SphK activity was required for TNFα-induced NF-κB activation (Xia et al., 1998, supra; Xia et al., 2002, J. Biol. Chem. 277:7996-8003). We therefore examined the effects of high glucose-induced SphK activity on NF-κB activation. Utilizing electrophoretic mobility shift assays we showed that incubation of HUVEC or BAEC with high glucose resulted in a significant nuclear NF-κB accumulation with preferentially DNA-p50 subunit complexes (FIG. 8). Remarkably, in the presence of the SphK-specific inhibitor DMS, high glucose-induced NF-κB activation was completely inhibited (Lane 3, FIG. 8). Furthermore, high glucose was incapable of activating NF-κB in the cells expressing SphK^{G82D} (Lane 8, FIG. 8). Together, these data indicate an important role for SphK in mediating high glucose-induced NF-κB activation in endothelial cells.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Beckman, J. A., Creager, M. A., and Libby, P. 2002. Diabetes and atherosclerosis: epidemiology, pathophysiology, and management. JAMA 287:2570-2581.
Bunin B A, et al. (1994) Proc. Natl. Acad. Sci. USA, 91:4708-4712
DeWitt S H, et al. (1993) Proc. Natl. Acad. Sci. USA, 90:6909-6913
Edsall, L. C., Van Brocklyn, J. R., Cuvillier, O., Kleuser, B., and Spiegel, S. 1998. N,N-dimethylsphingosine is a potent competitive inhibitor of sphingosine kinase but not of protein kinase C: modulation of cellular levels of sphingosine 1-phosphate and ceramide. Biochemistry 37:12892-12898.
Johnson, K. R., Becker, K. P., Facchinetti, M. M., Hannun, Y. A., and Obeid, L. M. 2002. PKC-dependent activation of sphingosine kinase 1 and translocation to the plasma membrane. Extracellular release of sphingosine-1-phosphate induced by phorbol 12-myristate 13-acetate (PMA). J. Biol. Chem. 277:35257-35262.
Morigi, M., Angioletti, S., Imberti, B., Donadelli, R., Micheletti, G., Figliuzzi, M., Remuzzi, A., Zoja, C., and Remuzzi, G. 1998. Leukocyte-endothelial interaction is augmented by high glucose concentrations and hyperglycaemia in a NF-κB-dependent fashion. J. Clin. Invest 101:1905-1915.
Pieper, G. M. and Riaz, u.H.1997. Activation of nuclear factor-kappaB in cultured endothelial cells by increased glucose concentration: prevention by calphostin C. J. Cardiovasc. Pharmacol. 30:528-532.
Pitson, S. M., D'Andrea, R. J., Vandeleur, L., Moretti, P. A., Xia, P., Gamble, J. R., Vadas, M. A., and Wattenberg, B. W. 2000. Human sphingosine kinase: purification, molecular cloning and characterization of the native and recombinant enzymes. Biochem. J. 350 Pt 2:429-441.
Pitson, S. M., Moretti, P. A., Zebol, J. R., Xia, P., Gamble, J. R., Vadas, M. A., D'Andrea, R. J., and Wattenberg, B. W. 2000. Expression of a catalytically inactive sphingosine kinase mutant blocks agonist-induced sphingosine kinase activation. A dominant-negative sphingosine kinase. J. Biol. Chem. 275:33945-33950.
Pitson, S. M., Moretti, P. A., Zebol, J. R., Lynn, H. E., Xia, P., Vadas, M. A., and Wattenberg, B. W. 2003. Activation of sphingosine kinase 1 by ERK1/2-mediated phosphorylation. EMBO J. 22:5491-5500.
Read, M. A., Whitley, M. Z., Williams, A. J., and Collins, T. 1994. NF-kappa B and I kappa B alpha: an inducible regulatory system in endothelial activation. J. Exp. Med. 179:503-512.
Sanchez, T. and Hla, T. 2004. Structural and functional characteristics of S1P receptors. J. Cell Biochem. 92:913-922.
The Diabetes Control and Complications Trial Research Group. 1993. The effect of intensive treatment of diabetes on the development and progression of long-term complications in insulin-dependent diabetes mellitus. N. Engl. J. Med. 329:977-986.
UK Prospective Diabetes Study (UKPDS) Group. 1998. Intensive blood-glucose control with sulphonylureas or insulin compared with conventional treatment and risk of complications in patients with type 2 diabetes (UKPDS 33). Lancet 352:837-853.
Van Brocklyn, J. R., Lee, M. J., Menzeleev, R., Olivera, A., Edsall, L., Cuvillier, O., Thomas, D. M., Coopman, P. J., Thangada, S., Liu, C. H. et al. 1998. Dual actions of sphingosine-1-phosphate: extracellular through the Gi-coupled receptor Edg-1 and intracellular to regulate proliferation and survival. J Cell Biol 142:229-240.
Verrier, E., Wang, L., Wadham, C., Albanese, N., Hahn, C., Gamble, J. R., Chatterjee, V. K., Vadas, M. A., and Xia, P. 2004. PPARgamma agonists ameliorate endothelial cell activation via inhibition of diacylglycerol-protein kinase C signaling pathway: role of diacylglycerol kinase. Circ. Res. 94:1515-1522.
Xia, P., Inoguchi, T., Kern, T. S., Engerman, R. L., Oates, P. J., and King, G. L. 1994. Characterization of the mechanism for the chronic activation of diacylglycerol-protein kinase C pathway in diabetes and hypergalactosemia. Diabetes 43:1122-1129.
Xia, P., Aiello, L. P., Ishii, H., Jiang, Z. Y., Park, D. J., Robinson, G. S., Takagi, H., Newsome, W. P., Jirousek, M. R., and King, G. L. 1996. Characterization of vascular endothelial growth factor's effect on the activation of protein kinase C, its isoforms, and endothelial cell growth. J. Clin. Invest 98:2018-2026
Xia, P., Gamble, J. R., Rye, K. A., Wang, L., Hii, C. S., Cockerill, P., Khew-Goodall, Y., Bert, A. G., Barter, P. J., and Vadas, M. A. 1998. Tumor necrosis factor-alpha induces adhesion molecule expression through the sphingosine kinase pathway. Proc. Natl. Acad. Sci. U.S.A 95:14196-14201.
Xia, P., Wang, L., Gamble, J. R., and Vadas, M. A. 1999. Activation of sphingosine kinase by tumor necrosis factor-alpha inhibits apoptosis in human endothelial cells. J. Biol. Chem. 274:34499-34505.
Xia, P., Wang, L., Moretti, P. A., Albanese, N., Chai, F., Pitson, S. M., D'Andrea, R. J., Gamble, J. R., and Vadas, M. A. 2002. Sphingosine kinase interacts with TRAF2 and dissects tumor necrosis factor-alpha signaling. J. Biol. Chem. 277:7996-8003.
Yang, N., Gene Transfer into Mammalian Somatic Cells in vivo, Grit. Rev. Biotech. 12(4):335-356 (1992)

1. A method of downregulating hyperglycaemia-induced endothelial cell functioning said method comprising downregulating sphingosine kinase mediated signalling in said cell.

2. A method of downregulating hyperglycaemia-induced endothelial cell functioning in a mammal said method comprising downregulating sphingosine kinase mediated signalling in said cell.

3. A method for the treatment and/or prophylaxis of a condition in a mammal, which condition is characterised by aberrant, unwanted or otherwise inappropriate hyperglycaemia-induced endothelial cell functioning said method comprising down-regulating sphingosine kinase mediated signalling in said cell.

4. The method according to claim 3 wherein said condition is diabetes, Cushing's disease, Cushing's syndrome, hyperthyroidism, metabolic syndrome or acromegalic.

5. A method of downregulating diabetes induced endothelial cell functioning in a mammal said method comprising downregulating sphingosine kinase mediated signalling in said cell.

6. The method according to claim 4 or 5 wherein said diabetes is Type 1 diabetes, Type 2 diabetes, gestational diabetes, slowly progressive adult onset IDDM or latent autoimmune diabetes in adults.

7. The method according to any one of claims 1 to 6 wherein said endothelial cell is a vascular endothelial cell.

8. The method according to claim 7 wherein said vascular endothelial cell functioning is vascular endothelial cell dysfunction.

9. The method according to claim 8 wherein said vascular endothelial cell dysfunction is vasculopathy.

10. The method according to claim 9 wherein said vasculopathy is microvasculopathy including lesions in microvascular beds of the retina, renal glomeruli or nerve tissues.

11. The method according to claim 9 wherein said vasculopathy is macrovasculopathy including lesions in the coronary or peripheral large blood vessels.

12. The method according to claim 9 wherein said vasculopathy is the upregulation of endothelial cell surface adhesion molecule expression, vascular inflammation, atherogenic lesions, increased endothelial permeability, abnormalities in vascular regeneration, contractility or blood flow or aberrant coagulation.

13. The method according to any one of claims 1 to 12 wherein said downregulation of sphingosine kinase mediated signalling is achieved by contacting said endothelial cell with a proteinaceous or non-proteinaceous molecule which functions as an antagonist to the sphingosine kinase expression product.

14. The method according to claim 13 wherein said antagonist is GF109203X, PD98059, U0126, N′N′-dimethylsphingosine or a mutant sphingosine kinase protein characterised by substitution of the G residue at position 82 with a D residue.

15. The method according to any one of claims 1 to 12 wherein said downregulation of sphingosine kinase mediated signalling is achieved by contacting said endothelial cell with a proteinaceous or non-proteinaceous molecule which down-regulates transcriptional and/or translational regulation of the sphingosine kinase gene.

16. The method according to any one of claims 1 to 12 wherein said downregulation of sphingosine kinase mediated signalling is achieved by contacting said endothelial cell with an antagonist of sphingosine-1-phosphate or an inhibitor of sphingosine-1-phosphate receptors which downregulates sphingosine-1-phosphate receptor functioning.

17. The method according to claim 16 wherein said antagonist is pertussis toxin.

18. The method according to any one of claims 1 to 12 wherein said downregulation of sphingosine kinase mediated signalling is achieved by downregulating the ability of glucose to induce activation of sphingosine kinase mediated signalling.

19.-36. (canceled)
